(19) United States
(12) Patent Application Publication
(10) Pub. No.: US 2024/0374628 A1
(43) Pub. Date: Nov. 14, 2024

(54) Triple Drug Combination (Metformin, Simvastatin, Digoxin) for Targeted Treatment of Colorectal Cancer

(71) Applicant: Whispergenics. Inc., West Hollywood, CA (US)

(72) Inventors: Francis Charles Brunicardi, West Hollywood, CA (US); Robbi L. Sanchez, West Hollywood, CA (US)

(21) Appl. No.: 18/693,693

(22) PCT Filed: Oct. 5, 2022
(86) PCT No.: PCT/US2022/045737

§ 371 Date: Mar. 20, 2024

Related U.S. Application Data
(60) Provisional application No. 63/252,859, filed on Oct. 6, 2021.

(51) Int. Cl. A61K 31/7048; A61K 31/155; A61K 31/282; A61K 31/366; A61K 31/4745; A61K 31/513; A61K 31/519; A61K 31/7068; A61P 35/00

(52) U.S. Cl. CPC A61K 31/7048; A61K 31/155; A61K 31/282; A61K 31/366; A61K 31/4745; A61K 31/513; A61K 31/519; A61K 31/7068; A61P 35/00

(57) ABSTRACT

The present invention includes a method of inhibiting growth of cancer in a patient, the method comprising contacting the cancer with amounts of metformin or a metformin analog; at least one statin selected from the group consisting of: simvastatin, atorvastatin, rosuvastatin, fluvastatin, lovastatin, pitavastatin, pravastatin, and prosuvastatin; and a cardiac glycoside in an amount sufficient to inhibit the growth of the breast, colorectal, glioblastoma or prostate cancer.

### TECHNICAL FIELD OF THE INVENTION

The present invention relates in general to the field of treatments for colorectal cancer, and more particularly, to the use of a triple drug combination (metformin, simvastatin, digoxin) for targeted treatment of breast, colorectal, glioblastoma or prostate cancer.

### STATEMENT OF FEDERALLY FUNDED RESEARCH

Not applicable.

### BACKGROUND OF THE INVENTION

Without limiting the scope of the invention, its background is described in connection with breast, colorectal, glioblastoma or prostate cancer.

One such invention is U.S. Pat. No. 11,096,947, issued to Tiedt, et al., entitled, “Combination products with tyrosine kinase inhibitors and their use” and is said to teach using a combination of (i) a MET inhibitor and (ii) an EGFR inhibitor, or a pharmaceutically acceptable salt thereof, respectively, or a prodrug thereof, which are jointly active in the treatment of proliferative diseases, corresponding pharmaceutical formulations, uses, methods, processes, commercial packages and related invention embodiments.

One such patent application is U.S. Patent Publication No. 20210292400, filed by Han, et al., and is entitled “Methods for treating, preventing and detecting the prognosis of colorectal cancer”, which is said to relate to treating cancer including colorectal cancer (CRC) by inhibiting or blocking Annexin 1, preventing CRC in a patient at high risk by inhibiting or blocking Annexin A1, reducing chemo-resistance by inhibiting or blocking Annexin A1, and detecting a poor prognosis in a subject with CRC by detecting or measuring the expression level of ANXA1 and/or the level of Annexin 1 protein.

Despite these improvements, there is a need in the art for improved methods and compositions for treating breast, colorectal, glioblastoma or prostate cancers.

### SUMMARY OF THE INVENTION

The invention disclosed herein meets this need using a combination of well-known and FDA approved therapeutic compounds having an unexpectedly potent therapy for colorectal cancers.

As embodied and broadly described herein, an aspect of the present disclosure relates to a method of inhibiting growth of cancer in a patient, the method comprising contacting the cancer with a combination therapy that comprises: a metformin or a metformin analog; at least one statin selected from the group consisting of: simvastatin, atorvastatin, rosuvastatin, fluvastatin, lovastatin, pitavastatin, pravastatin, and prosuvastatin; and a cardiac glycoside, in amounts sufficient to inhibit the growth of the cancer, wherein the cancer is selected from breast, colorectal, glioblastoma or prostate cancer. In one aspect, the method further comprises a pharmaceutically acceptable carrier selected to be compatible with metformin, simvastatin, and digoxin. In another aspect, wherein the combination therapy is a composition formulated as a time release formulation. In another aspect, the combination therapy is a composition is disposed in a capsule, a tablet, a powder, or a liquid. In another aspect, the combination therapy comprises: 5-80 milligrams of simvastatin; 500-2550 milligrams of metformin; and/or 0.125-0.250 milligrams of digoxin.

In another aspect, the metformin, simvastatin and digoxin are combined into one or more doses before providing to the patient with breast, colorectal, glioblastoma or prostate cancer. In another aspect, the method further comprises observing the breast, colorectal, glioblastoma or prostate cancer for evidence of reduced tumor size, inhibition of tumor growth, slowing of tumor growth, or tumor death following exposure to the metformin, simvastatin and digoxin. In another aspect, the colorectal cancer is selected from at least one of: familial polyposis, polyps with DNA mismatch repair, MSI, Lynch Syndrome, adenomatous polyps, or sessile serrated polyps. In another aspect, the breast, colorectal, glioblastoma or prostate cancer is a metastatic cancer. In another aspect, the patient is administered 500-2550 milligrams/day of metformin, 5-80 milligrams/day of simvastatin, and 0.125-0.250 milligrams/day of digoxin; or 5-80 milligrams per os (po) of simvastatin; 500-2550 milligrams po of metformin; and 0.125-0.250 milligrams po of digoxin. In another aspect, the method further comprises combining the composition with effective amounts of at least one of Bevacizumab, Irinotecan Hydrochloride, Capecitabine, Cetuximab, Ramucirumab, Oxaliplatin, Cetuximab, 5-Fluorouracil, Ipilimumab, Irinotecan Hydrochloride, Pembrolizumab, Leucovorin Calcium, Trifluridine and Tipiracil Hydrochloride, Nivolumab, Oxaliplatin, Panitumumab, Ramucirumab, Regorafenib, Trifluridine and Tipiracil Hydrochloride, Ziv-Aflibercept, or drug combinations selected from: CAPOX, FOLFIRI, FOLFIRI-BEVACIZUMAB, FOLFIRI-CETUXIMAB, FOLFOX, FU-LV, XELIRI, or XELOX.

As embodied and broadly described herein, an aspect of the present disclosure relates to a method treating a cancer identified as expressing BIRC5 mRNA, the method comprising providing a human patient with an effective amount of metformin, simvastatin and digoxin sufficient to inhibit the expression of BIRC5 mRNA or BIRC5 activity in the cancer, wherein the amounts of metformin, simvastatin, and digoxin sufficient to inhibit in vivo growth of a human cancer when administered to the patient diagnosed with cancer, wherein the cancer is selected from breast, colorectal, glioblastoma or prostate cancer. In one aspect, the method further comprises a pharmaceutically acceptable carrier selected to be compatible with metformin, simvastatin, and digoxin. In another aspect, the metformin, simvastatin and digoxin are formed as a time release formulation. In another aspect, the metformin, simvastatin and digoxin are disposed in a capsule or tablet. In another aspect, the amounts of metformin, simvastatin, and digoxin are sufficient to inhibit in vivo growth of a human breast, colorectal, glioblastoma or prostate cancer cell when administered to a patient diagnosed with breast, colorectal, glioblastoma or prostate cancer. In another aspect, the metformin, simvastatin and digoxin comprises: 5-80 milligrams of simvastatin; 500-2550 milligrams of metformin; and/or 0.125-0.250 milligrams of digoxin; or 5-80 milligrams per os (po) of simvastatin; 500-2550 milligrams po of metformin; and 0.125-0.250 milligrams po of digoxin. In another aspect, the metformin, simvastatin and digoxin are combined into one or more doses before providing to the patient with breast, colorectal, glioblastoma or prostate cancer. In another aspect, the method further comprises observing the breast, colorectal, glioblastoma or prostate cancer for evidence of reduced tumor size, inhibition of tumor growth, slowing of tumor growth, or tumor death following exposure to the metformin, simvastatin and digoxin. In another aspect, the colorectal cancer is selected from at least one of: familial polyposis, polyps with DNA mismatch repair, MSI, Lynch Syndrome, adenomatous polyps, or sessile serrated polyps. In another aspect, the breast, colorectal, glioblastoma or prostate cancer is a metastatic cancer. In another aspect, the patient is administered 500-2550 milligrams/day of metformin, 5-80 milligrams/day of simvastatin, and 0.125-0.250 milligrams/day of digoxin. In another aspect, the method further comprises combining a population of colorectal cancer cells with amounts of at least one of Bevacizumab, Irinotecan Hydrochloride, Capecitabine, Cetuximab, Ramucirumab, Oxaliplatin, Cetuximab, 5-Fluorouracil, Ipilimumab, Irinotecan Hydrochloride, Pembrolizumab, Leucovorin Calcium, Trifluridine and Tipiracil Hydrochloride, Nivolumab, Oxaliplatin, Panitumumab, Ramucirumab, Regorafenib, Trifluridine and Tipiracil Hydrochloride, Ziv-Aflibercept, or drug combinations selected from: CAPOX, FOLFIRI, FOLFIRI-BEVACIZUMAB, FOLFIRI-CETUXIMAB, FOLFOX, FU-LV, XELIRI, or XELOX. In another aspect, the metformin, simvastatin and digoxin is combined with a plurality of cells in an amount sufficient to promote cellular apoptosis in the human breast, colorectal, glioblastoma or prostate cancer. In another aspect, the method further comprises treating the patient with gemcitabine.

As embodied and broadly described herein, an aspect of the present disclosure relates to a method treating a cancer in a human patient identified as expressing BIRC5 mRNA, the method comprising: determining that cancer cells in a subject have an overexpression, or increased activity, of BIRC5 when compared to a subject that does not have cancer; and providing the patient with an effective amount of metformin, simvastatin and digoxin sufficient to inhibit the expression of BIRC5 mRNA or BIRC5 activity in the cancer, wherein the amounts of metformin, simvastatin, and digoxin sufficient to inhibit in vivo growth of a human cancer when administered to the patient diagnosed with cancer, wherein the cancer is selected from breast, colorectal, glioblastoma or prostate cancer. In one aspect, the method further comprises a pharmaceutically acceptable carrier selected to be compatible with metformin, simvastatin, and digoxin. In another aspect, the metformin, simvastatin and digoxin are formed as a time release formulation. In another aspect, the metformin, simvastatin and digoxin are disposed in a capsule or tablet. In another aspect, the amounts of metformin, simvastatin, and digoxin are sufficient to inhibit in vivo growth of a human breast, colorectal, glioblastoma or prostate cancer cell when administered to a patient diagnosed with breast, colorectal, glioblastoma or prostate cancer. In another aspect, the metformin, simvastatin and digoxin comprises: 5-80 milligrams of simvastatin; 500-2550 milligrams of metformin; and/or 0.125-0.250 milligrams of digoxin; or 5-80 milligrams per os (po) of simvastatin; 500-2550 milligrams po of metformin; and 0.125-0.250 milligrams po of digoxin. In another aspect, the metformin, simvastatin and digoxin are combined into one or more doses before providing to the patient with breast, colorectal, glioblastoma or prostate cancer. In another aspect, the method further comprises observing the breast, colorectal, glioblastoma or prostate cancer for evidence of reduced tumor size, inhibition of tumor growth, slowing of tumor growth, or tumor death following exposure to the metformin, simvastatin and digoxin. In another aspect, the colorectal cancer is selected from at least one of: familial polyposis, polyps with DNA mismatch repair, MSI, Lynch Syndrome, adenomatous polyps, or sessile serrated polyps. In another aspect, the breast, colorectal, glioblastoma or prostate cancer is a metastatic cancer. In another aspect, the patient is administered 500-2550 milligrams/day of metformin, 5-80 milligrams/day of simvastatin, and 0.125-0.250 milligrams/day of digoxin. In another aspect, the method further comprises combining a population of colorectal cancer cells with amounts of at least one of Bevacizumab, Irinotecan Hydrochloride, Capecitabine, Cetuximab, Ramucirumab, Oxaliplatin, Cetuximab, 5-Fluorouracil, Ipilimumab, Irinotecan Hydrochloride, Pembrolizumab, Leucovorin Calcium, Trifluridine and Tipiracil Hydrochloride, Nivolumab, Oxaliplatin, Panitumumab, Ramucirumab, Regorafenib, Trifluridine and Tipiracil Hydrochloride, Ziv-Aflibercept, or drug combinations selected from: CAPOX, FOLFIRI, FOLFIRI-BEVACIZUMAB, FOLFIRI-CETUXIMAB, FOLFOX, FU-LV, XELIRI, or XELOX. In another aspect, the metformin, simvastatin and digoxin is combined with a plurality of cells in an amount sufficient to promote cellular apoptosis in the human breast, colorectal, glioblastoma or prostate cancer. In another aspect, the method further comprises treating the patient with gemcitabine.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the viability of three human colon cancer cell lines to different amounts of Digoxin. It was shown that HT29 is very sensitive to Digoxin.

FIG. 2 is a graph that shows the viability of three human colon cancer cell lines to different amounts of Simvastatin. It was shown that CCD-18Co is very sensitive to Simvastatin.

FIG. 3 is a graph that shows the viability of three human colon cancer cell lines to different amounts of Metformin. It was shown that Caco2 is sensitive to Metformin.

FIG. 4 is a graph that shows a dose curve for the combination of Digoxin, Simvastatin, and Metformin (C3) at C3: 50 nM Dig/5 um Sim/2.5 mM Met inhibits human colon cancer cell line viability in all cases.

FIG. 5 is a graph that shows that the combination of Digoxin, Simvastatin, and Metformin (C3) inhibits human colon cancer cell line viability in all cases showing relative cell viability (%). C3: 50 nM Dig/5 um Sim/2.5 mM Met.

FIG. 6 is a graph that shows that C3 therapy inhibits BIRC5 promoter activity in MDA 231 Breast Cancer Cells.

FIG. 7 is a graph that shows the reduced viability of MDA 231 Breast Cancer Cells after C3 therapy.

FIG. 8 is a graph that shows that C3 therapy inhibits BIRC5 promoter activity in U87 Glioblastoma Cells.

FIG. 9 is a graph that shows the reduced viability of U87 Glioblastoma Cells after C3 therapy.

FIG. 10 is a graph that shows that C3 therapy inhibits BIRC5 promoter activity in U251 Glioblastoma Cells.

FIG. 11 is a graph that shows the reduced viability of U251 Glioblastoma Cells after C3 therapy.

FIG. 12 is a graph that shows that C3 therapy inhibits BIRC5 promoter activity in Du145 Prostate Cancer Cells.

FIG. 13 is a graph that shows the reduced viability of Du145 Prostate Cancer Cells after C3 therapy.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The composition comprises amounts of agents such as metformin, simvastatin, and digoxin (C3) that are sufficient to inhibit in vivo growth of colorectal cancer when administered orally to a patient diagnosed with colorectal cancer, or a metastatic colorectal cancer. In one example, a patient is one year out on C3 and had metastatic colorectal cancer, which was refractory to all other therapies. In certain non-limiting examples, the colorectal cancer is strongly positive for BIRC5 and PDX-1.

The present inventors have previously explored the effects of a triple combination treatment in pancreatic cancer. For example, U.S. Pat. No. 10,813,909, issued to Brunicardi, et al., entitled “Triple drug combination (metformin, simvastatin, digoxin) for targeted treatment of pancreatic cancer”, teaches that three well-known and FDA approved compounds has been discovered to significantly suppress the proliferation of pancreatic cancer cells in clinically relevant models of pancreatic cancer. The inventors teach compositions of matter comprising a combination of agents such as metformin, simvastatin, and digoxin as well as methods of treating cancers using such agents. Illustrative methods include combining a population of pancreatic cancer cells with amounts of metformin, simvastatin, and digoxin sufficient to inhibit expression of BIRC5 protein in the population of pancreatic cancer cells, thereby inhibiting the growth of the population of pancreatic cancer cells.

As disclosed in detail below, and surprisingly, it has been discovered that a combination of the three well-known and FDA approved drugs, metformin, simvastatin, and digoxin, can inhibit human colorectal cancer cell growth in vivo.

The data presented herein show that a triple drug combination comprising metformin or a metformin analog, a statin such as simvastatin, and a cardiac glycoside such as digoxin can inhibit colorectal cancer growth by inhibiting genes involving cell proliferation and energy metabolism, and the predominant drug in these effects are the statins. Without being bound by a specific theory or mechanism of action, the data presented herein provides evidence that this triple drug combination acts to cause cancer cell death via promotion of apoptosis genes via synergistic effects of the three drugs. Experimental data further demonstrates that the triple drug combination inhibits a network of cancer cell proliferation genes, in particular BIRC5.

The invention disclosed herein has a number of embodiments. In one embodiment of the invention, a composition of matter is provided comprising a combination of metformin or metformin analog, a statin, and a cardiac glycoside. In the typical working embodiments of the invention that are disclosed herein, this composition of matter comprises a combination of metformin, simvastatin, and digoxin. Metformin, simvastatin, and digoxin are all therapeutic compounds that have been approved by the U.S. Food and Drug Administration (FDA). Metformin is typically administered for the treatment of type 2 diabetes. Simvastatin is typically administered for the treatment of elevated lipid levels (e.g., low-density lipoprotein, triglycerides) and to lower the risk of stroke, heart attack, and other heart complications. Digoxin is typically administered for the treatment of heart failure and atrial fibrillation. Unexpectedly, a composition comprising the combination of these three therapeutic compounds provides an effective adjuvant therapy for colorectal cancer.

Embodiments of the invention include compositions of matter comprising at least two of the following three therapeutic agents: a biguanide such as metformin (or metformin analog), a statin, and a cardiac glycoside. Optionally this composition can include one or more additional agents such as another therapeutic agent approved for the treatment of colorectal cancer. Additional agents can also include other therapeutic agent approved for other uses, for example a drug identified in Tables 5A-5C below, or a sulfonylurea such as acetohexamide, carbutamide, chlorpropamide, glycyclamide (tolhexamide), metahexamide, tolazamide tolbutamide, glibenclamide (glyburide), glibornuride, gliclazide, glipizide, gliquidone, glisoxepide, glyclopyramide and glimepiride. Embodiments of the invention include those where the dosages of such therapeutic agents are within the range approved for use of that agent in humans by the Food and Drug Administration (as found, for example in databases such as “Drugs@FDA: FDA Approved Drug Products”). In one illustrative embodiment of this composition, the composition is in the form of a pill or tablet (including a plurality of pills or tablets) or the like and comprises a daily (or weekly or monthly) dose of those agents that is within the ranges approved for use of those agents in humans by the Food and Drug Administration.

While simvastatin, metformin and digoxin are illustrative working embodiments in this disclosure, it is to be noted that metformin, simvastatin, and digoxin may be substituted with other metformin analogs, statins, and cardiac glycosides, respectively, in one or more embodiments of the invention. Statins such as simvastatin are HMG-CoA reductase inhibitors. Data from studies with statins that are presented herein provide evidence that HMG-CoA reductase inhibitors are useful in embodiments of the invention. Illustrative statins useful in embodiments of the invention include Lipitor (atorvastatin), Lescol (fluvastatin), Mevacor (lovastatin), Altoprev (lovastatin extended-release), Livalo (pitavastatin), Pravachol (pravastatin), Crestor (rosuvastatin), and Zocor (simvastatin), cerivastatin and mevastatin.

Metformin is a first-line medication for the treatment of type 2 diabetes, particularly in people who are overweight. Metformin is also used in the treatment of polycystic ovary syndrome. Illustrative metformin analogs include the analogs as described in Pietras et al. (PCT Application No. PCT/US2013/045250). Digoxin is in the cardiac glycoside family of medications. Data from studies with digoxin that are presented herein provide evidence that cardiac glycosides are useful in embodiments of the invention. Cardiac glycosides are a class of organic compounds that affect the inotropic and chronotropic activity of the heart by acting on the sodium-potassium ATPase pump. These cardiac glycosides are Na+/K+ ATPase inhibitors that act via the Warburg effect. Bufalin, ouabain and digoxin are a few illustrative cardiac glycosides. Digitalis is another commonly used cardiac glycoside. Digoxin preparations are marketed under the trade names Cardigox; Cardiogoxin; Cardioxin; Cardoxin; Coragoxine; Digacin; Digicor; Digomal; Digon; Digosin; Digoxine Navtivelle; Digoxina-Sandoz; Digoxin-Sandoz; Digoxin-Zori; Dilanacin; Eudigox; Fargoxin; Grexin; Lanacordin; Lanacrist; Lanicor; Lanikor; Lanorale; Lanoxicaps; Lanoxin; Lanoxin PG; Lenoxicaps; Lenoxin; Lifusin; Mapluxin; Natigoxin; Novodigal; Purgoxin; Sigmaxin; Sigmaxin-PG; Toloxin. Using experimental studies such as those disclosed herein, we have identified a number cardiac glycosides that inhibit BIRC5 expression in colorectal cancer (as well as similarly regulated genes). These cardiac glycosides include digoxin, digitoxigen, digoxigen, digitalis, lanatoside C, bufalin and oubain.

Typically, the compositions of the invention are used to modulate the growth of colorectal cancer cells that express BIRC5 protein/mRNA. Colorectal cancers or neoplasms of the colorectal tract include neoplasms, such as familial polyposis, polyps with DNA mismatch repair, MSI, Lynch Syndrome, adenomatous polyps and sessile serrated polyps.

In one or more embodiments of the invention, the composition is used to inhibit the growth of a colorectal neoplasms. In certain embodiments of the invention, the composition comprises these three agents in combination with a colorectal cancer cell.

In typical embodiments of the invention, the composition comprises amounts of metformin, simvastatin, and digoxin sufficient to inhibit/suppress in vivo growth of a colorectal cancer cell when administered to a patient diagnosed with colorectal cancer. In one embodiment, the composition comprises amounts of metformin, simvastatin, and digoxin sufficient to inhibit/suppress in vivo growth of a human colorectal cancer cell when orally administered to a patient diagnosed with colorectal cancer. Typically, the composition comprises 5-80 milligrams per os (po) of simvastatin; 500-2550 milligrams po of metformin; and 0.125-0.250 milligrams po of digoxin. In some embodiments, the composition can comprise from 62.5 micrograms to 500 micrograms po of digoxin. The composition of matter typically further comprises a pharmaceutically acceptable carrier. In one or more embodiments, the composition is formed as a time release formulation and may be disposed in a capsule or tablet.

The fact that statins, metformin and digoxin are all well-known drugs that have been used in patients for years to treat other syndromes/diseases allows information from both current and previous studies on the dose and efficacy of these agents to be used to identify doses for use the triple drug therapies disclosed herein (e.g. as a treatment for colorectal cancer). For example, in embodiments of the invention, the in vivo dose of simvastatin can be approximately 20 mg/kg (e.g., from 10 mg/kg to 30 mg/kg), the in vivo dose of digoxin can be approximately 2 mg/kg (e.g., from 1 mg/kg to 3 mg/kg), and the in vivo dose of metformin can be approximately 100 mg/kg (e.g., from 50 mg/kg to 150 mg/kg). In embodiments of the invention, the human clinical dose of simvastatin can be ˜80 mg/day orally, so the human dose in this embodiment of the invention is ˜1.14 mg/kg. In embodiments of the invention, the level of digoxin for treatment is typically 0.5-2 ng/mL. Since this is a narrow therapeutic index, it is therefore important that digoxin concentration be maintained in approximately this range if it is used in patients with other conditions such as heart failure.

While it is possible for the combination of active ingredients of the composition to be administered without other ingredients, it is preferable to present them within a pharmaceutical formulation. Pharmaceutical formulations according to the present invention comprise the active ingredients (i.e., metformin, simvastatin, and digoxin) together with one or more pharmaceutically acceptable carriers or excipients and optionally other therapeutic agents. The pharmaceutically acceptable carrier(s) cannot be water alone and must be acceptable in the sense of being compatible with the other ingredients of the formula. In embodiments of the invention, the composition comprises a pharmaceutically acceptable carrier selected to be compatible with metformin and compatible simvastatin, and compatible digoxin when all three are combined in a single composition.

Illustrative formulations include those suitable for oral, enteral, topical (including transdermal, buccal and sublingual) or parenteral (including subcutaneous, intramuscular, intravenous, and intradermal) administration. The formulations may be prepared by any methods well known in the art of pharmacy, for example, using methods such as those described in Remington: The Science and Practice of Pharmacy (22nd ed., Pharmaceutical Press, 2012, see especially Section 5: Pharmaceutical Dosage Forms: Manufacturing and Compounding). Such methods include the step of bringing into association the active ingredient with the carrier which constitutes one or more accessory ingredients. Such accessory ingredients may include pharmaceutically acceptable auxiliary substances as required to, for example, stabilize the formulation and/or approximate physiological conditions. Illustrative agents include those conventional in the art, such as agents that inhibit microbial growth, pH adjusting and buffering agents, tonicity adjusting agents, wetting agents, detergents and the like, as well as fillers, binders, diluents, disintegrants, lubricants, colorants, flavoring agents and the like.

Formulations suitable for oral administration may be presented as discrete units such as pills, tablets or capsules each containing a predetermined amount of the active ingredients; as a powder or granules; as a solution or suspension. The active ingredients may also be present as a bolus or paste, or may be contained within liposomes. For parenteral administration, suitable formulations include aqueous and non-aqueous sterile injection. The formulations may be presented in unit-dose or multi-dose containers, for example, sealed vials and ampoules, and may be stored in a freeze dried (lyophilized) condition requiring only the addition of the sterile liquid carrier, for example, water prior to use.

In addition to the triple drug combination of metformin, simvastatin, and digoxin, the composition/methods may further include therapeutic compounds/regimens commonly used in first-line and/or second-line treatments for colorectal cancer. In one or more embodiments, the composition further includes a gemcitabine (gemzar), 5-fluorouracil (5-FU), irinotecan (camptosar), oxaliplatin (eloxatin), albumin-bound paclitaxel (abraxane), capecitabine (xeloda), cisplatin, paclitaxel (taxol), docetaxel (taxotere), irinotecan liposome (onivyde), or combinations thereof, including FOLFOX (folinic acid, fluorouracil, oxaliplatin) and FOLFIRINOX (folinic acid, fluorouracil, irinotecan, oxaliplatin).

In addition to using them in therapeutic regimens designed to treat colorectal cancer, the triple drug combinations disclosed herein are useful in a number of other contexts, for example in in vitro assays that are useful for examining cellular growth and differentiation. For example, BIRC5/Survivin is a member of the inhibitor of apoptosis (IAP) family and the survivin protein functions to inhibit caspase activation, thereby leading to negative regulation of apoptosis or programmed cell death. The BIRC5 protein is expressed highly in most human tumors and fetal tissue, but is absent in terminally differentiated cells. In this context, by targeting BIRC5, the triple drug compositions of the invention are useful in assays designed to characterize the state of differentiation of cells, for example an assay which combines a triple drug composition with a population of cells having an unknown differentiation state and then observing the level of growth inhibition caused by this combination. In one illustrative embodiment of such assays, a population of precancerous of cancerous or cancerous cells (e.g. cells obtained from a patient biopsy) is combined with the triple drug composition and the level of apoptosis in the presence of the three drugs is then observed in order to obtain information on the differentiation state of these cells.

Yet another embodiment of the invention is a method of inhibiting growth of a population of cells that express BIRC5 protein (UniProtKB: 015392). The method comprises combining the population of cells such as breast, colorectal, glioblastoma or prostate cancer cells with amounts of metformin, simvastatin, and digoxin sufficient to inhibit expression of BIRC5 protein in the population of cells, thereby inhibiting the growth of the population of cells. In specific instances, breast, colorectal, glioblastoma or prostate cancer cells are treated. In one or more of these embodiments of the invention, the population of cells are combined with metformin, simvastatin, and digoxin in vivo in a patient diagnosed with a disease syndrome such as breast, colorectal, glioblastoma or prostate cancer. In some embodiments, the method further comprises combining the population of breast, colorectal, glioblastoma or prostate cancer cells with amounts of at least one of a gemcitabine (gemzar), paclitaxel (abraxane), A23187 (calcimycin) or ouabain.

Optionally the method further comprises observing the population of cells for evidence of cell growth inhibition or cell death following exposure to the metformin, simvastatin, and digoxin.

A related embodiment of the invention is a method of inhibiting the expression of BIRC5 mRNA (Entrez Gene: 332) in a population of cells identified as expressing BIRC5 mRNA. An example of this method comprises combining the population of human cells with amounts of metformin, simvastatin, and digoxin sufficient to inhibit the expression of BIRC5 mRNA in the population of human cells. In one or more embodiments, the metformin, simvastatin, and digoxin are combined with a plurality of cells in an amount sufficient to promote apoptosis in the population of human cells. In one embodiment, the population of human cells are combined with metformin, simvastatin and digoxin in vivo. In another embodiment, the population of human cells are combined with metformin, simvastatin and digoxin in vitro. In some embodiments, the method further comprises observing the population of human cells for evidence of cell death. In specific instances, the population of human cells are colorectal cancer cells.

In certain embodiments, the patient is administered metformin, simvastatin, and digoxin using the composition of matter comprising a combination of metformin, simvastatin, and digoxin, wherein the composition is disposed in a capsule or tablet as a time release formulation. Preferably, the composition is administered to a patient orally. In other embodiments, the composition may be administered through other routes, such as enteral, parenteral, intravenous, and intraperitoneal administrations. In one specific implementation, the composition is given orally once per day indefinitely.

As discussed in detail herein, embodiments of the invention include compositions comprising a combination of a statin such as simvastatin, metformin, and a cardiac glycoside such as digoxin for use as a medicament. One illustrative example of this is a combination of metformin, simvastatin, and digoxin for use in the treatment of a cancer such as a colorectal cancer. A related embodiment is the use of a statin such as simvastatin, metformin, and a cardiac glycoside such as digoxin for the manufacture of a medicament for the treatment of a cancer such as a colorectal cancer.

Suitable dosages, preferably unit dosages, of the composition include the known permissible doses for these compounds separately as described or referred to in reference texts such as the British and US Pharmacopoeias, Remington: The Science and Practice of Pharmacy (Pharmaceutical Press), and Martindale: The Complete Drug Reference (Pharmaceutical Press). The dosages of each particular active agent in any given composition can as required vary within a range of doses known to be required in respect to accepted dosage regimens for that compound. Generally, the therapeutic compounds are administered to the patient in doses that are much lower than their median lethal doses, LD₅₀.

### TABLES 1A-1C

TABLE 1A
Drugs observed to inhibit expression from the BIRC5 promoter.

| Name of Drugs | Category of Drugs |
|---|---|
| Paclitaxel | Chemotherapy |
| Mitoxantrone dihydrochloride | |
| Daunorubicin hydrochloride | |
| Camptothecine (S, +) | |
| Cantharidin | |
| Thapsigargin | Cardiovascular |
| Lanatoside C | |
| Ouabain | |
| Digitoxigenin | |
| Fluvastatin sodium salt | |
| Lovastatin | |
| Niguldipine | |
| A-23187 | Antibiotics |
| Antimycin A | |
| Bergenin Monohydrate | Metabolism |

TABLE 1B
Drugs observed to inhibit expression from the SHIP promoter.

| Name of Drugs | Category of Drugs |
|---|---|
| Doxorubicin hydrochloride | Chemotherapy |
| Mitoxantrone dihydrochloride | |
| Daunorubicin hydrochloride | |
| Camptothecine (S, +) | |
| Proscillaridin A | Cardiovascular |
| Quabain | |
| Lanatoside C | |
| Digoxin | |
| Digitoxigenin | |
| Fluvastatin sodium salt | |
| Simvastatin | |
| Ethacrynic acid | |
| A-23187 | Antibiotics |
| Thiostrepton | |
| Metformin hydrochloride | Diabetes |

TABLE 1C
Drugs observed to inhibit expression from the LAMC2 promoter.

| Drug Name | Category |
|---|---|
| Danunorubicin hydrochloride | Chemodrug |
| Mitoxantrone | |
| dihydrochloride | |
| Doxorubicin hydrochloride | |
| Paclitaxel | |
| Camptothecine (S, +) | |
| Podophyllotoxin | Antitumor Derivatives include etoposide, |
| | teniposide, and etopophos |
| Proscillaridin A | Cardiovascular Drugs |
| Digoxin | |
| Digitosigenin | |
| Simvastatin | |
| Lovastatin | |
| Digoxigenin | |
| Bergenin monohydrate | |
| Fluvastatin sodium salt | |
| Lanatoside C | |
| ANTIBIOTIC A-23187 | Antibiotics |
| Cycloheximide | |

Cell lines: Human colon cancer cell lines CCD-18Co and Caco2 (American Type Culture Collection [ATCC], Bethesda, MD) were maintained in Eagle's Minimum Essential Medium (Thermo Fisher Scientific, MA) supplemented with 100,000 U/L of penicillin, 100,000 μg/L of streptomycin, and 20% fetal bovine serum (FBS). HT29 (American Type Culture Collection [ATCC], Bethesda, MD) was maintained in McCoy's 5a Medium Modified (Thermo Fisher Scientific, MA) supplemented with 100,000 U/L of penicillin, 100,000 μg/L of streptomycin, and 10% fetal bovine serum (FBS). The following cells were also tested: MDA 231 Breast Cancer Cells, U87 Glioblastoma Cells, U251 Glioblastoma Cells and Dul45 Prostate Cancer Cells. All cells were maintained in Eagles Medium as described above.

C3 treatment and cell viability the cells were growing in 96 well plates for 24 h followed by treatments with Digoxin (12.5 nM-800 nM), Simvastatin (1.25 μM-80 μM), Metformin (625 μM-40000 μM) and triple combination for 48 h. The cells were subjected to cell viability assays using The CellTiter-Glo luminescent cell viability kit (Promega, catalog number: G7570). luminescence was measured using microplate luminometer (luminoskan™ ascent, Thermo-Fisher Scientific, MA). Background luminescence was measured in medium without cells and subtracted from experimental values.

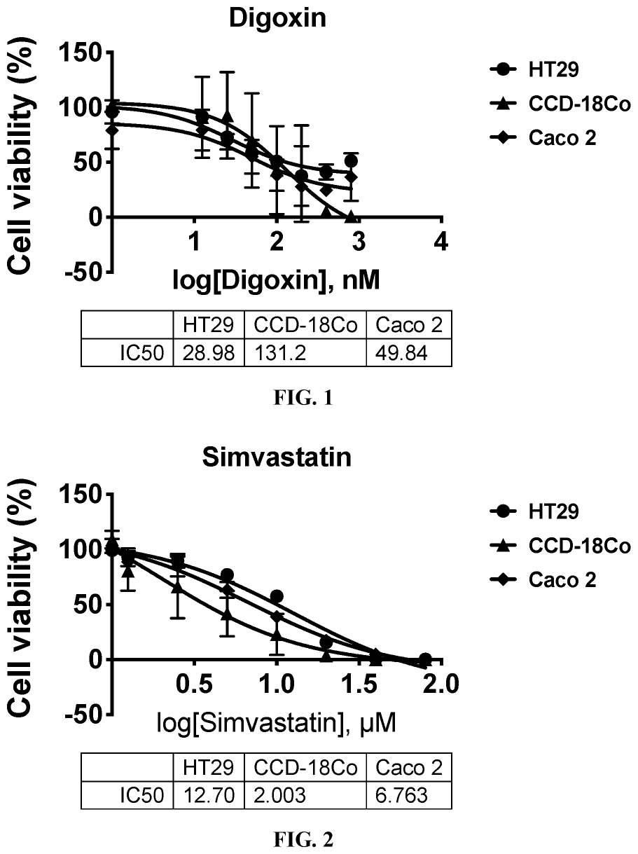

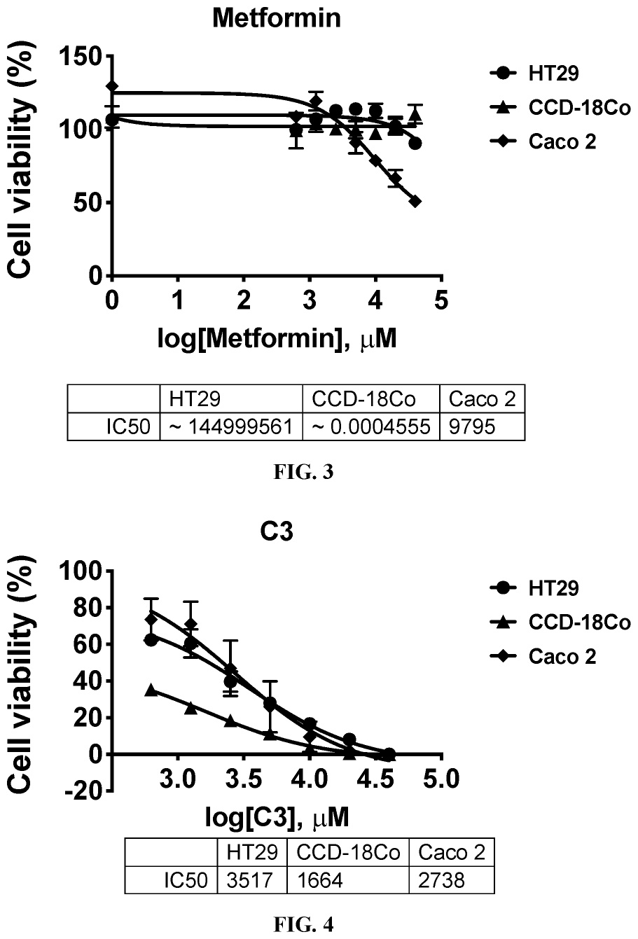

Results: Dose-response curves demonstrated that digoxin, simvastatin, or metformin alone reduced cell viability to varying degrees on human colon cancer cell lines (FIGS. 1, 2 and 3, respectively). The cell's response to each compound was evaluated by IC50 showing variation of the drug responses in different human colon cancer cell lines (FIGS. 1, 2 and 3, respectively). These results shows that single drug alone is insufficient to inhibit cell growth of all kinds of colon cancer cell lines. FIG. 1 shows the viability of three human colon cancer cell lines to different amounts of Digoxin. It was shown that HT29 is very sensitive to Digoxin. FIG. 2 shows the viability of three human colon cancer cell lines to different amounts of Simvastatin. It was shown that CCD-18Co is very sensitive to Simvastatin. FIG. 3 shows the viability of three human colon cancer cell lines to different amounts of Metformin. It was shown that Caco2 is sensitive to Metformin.

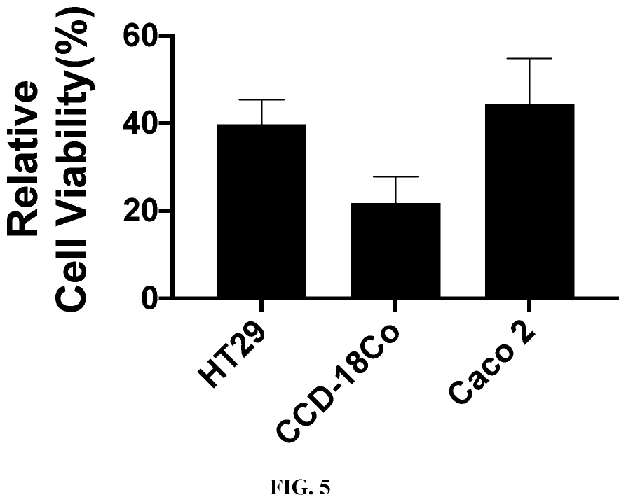

A combination of 50 nM digoxin, 5 μM simvastatin and 2.5 mM metformin (C3) was determined based on IC50 and applied colon cancer cells. The results demonstrated a significantly reduced cell viability of all 3 human colon cancer cells through addition, synergy, or complementary effect (p<0.05) (FIGS. 4 and 5). FIG. 4 is a graph that shows a dose curve for the combination of Digoxin, Simvastatin, and Metformin (C3) at C3: 50 nM Dig/5 um Sim/2.5 mM Met inhibits human colon cancer cell line viability in all cases. FIG. 5 shows that the combination of Digoxin, Simvastatin, and Metformin (C3) inhibits human colon cancer cell line viability in all cases showing relative cell viability (%). C3: 50 nM Dig/5 um Sim/2.5 mM Met.

Treatment of Colorectal Cancer with C3.

Interventions. The Phase IB trial of metformin, digoxin, and simvastatin in subjects with advanced pancreatic cancer and other advanced solid tumors was an open label, single center, single arm dose escalation study. C3 (metformin, simvastatin, and digoxin) was given as three oral pills. The subjects were accrued in 3-subject dose escalation cohorts. Cohort 1 received metformin 850 mg po/day, for two weeks, simvastatin 5 mg po/day, digoxin 0.0625 mg po/day. Cohort 2 received metformin 850 mg po/day, for two weeks and 1,700 mg po/day for the next two weeks, simvastatin 20 mg po/day, digoxin 0.25 mg po/day. Metformin was to be taken at breakfast and dinner, simvastatin at bedtime, and digoxin in the morning. The patients received metformin 850 mg po/day for two weeks and 1,700 mg po/day for the next two weeks, simvastatin 20 mg po/day, and digoxin 0.25 mg.

The three medications, metformin, digoxin, and simvastatin were selected for their ability to synergistically reduce BIRC5 expression. A subpopulation that responds positively to C3 treatment may be patients with breast, colorectal, glioblastoma, and prostate cancers who have high levels of BIRC5 expression.

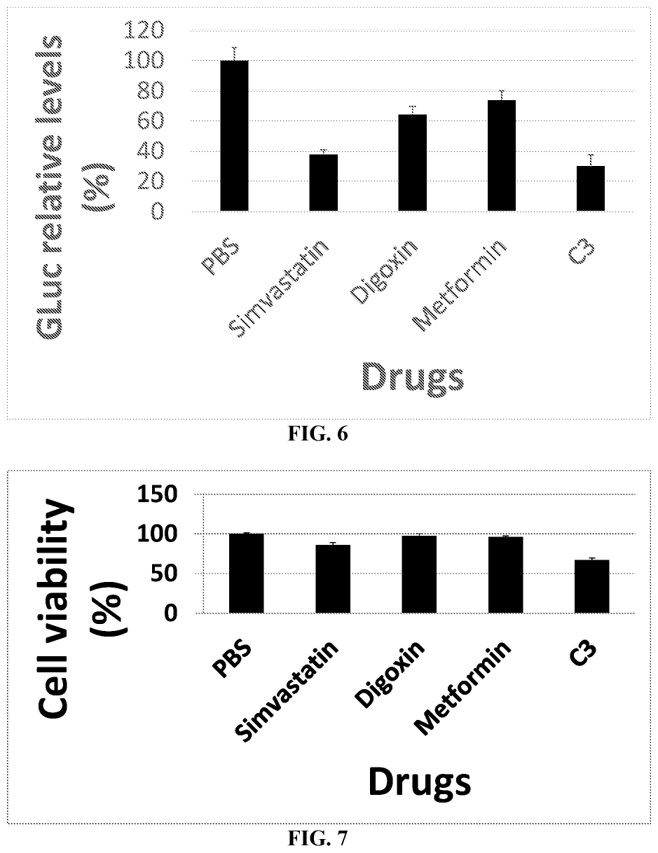

FIG. 6 is a graph that shows that C3 therapy inhibits BIRC5 promoter activity in MDA 231 Breast Cancer Cells. FIG. 7 is a graph that shows the reduced viability of MDA 231 Breast Cancer Cells after C3 therapy.

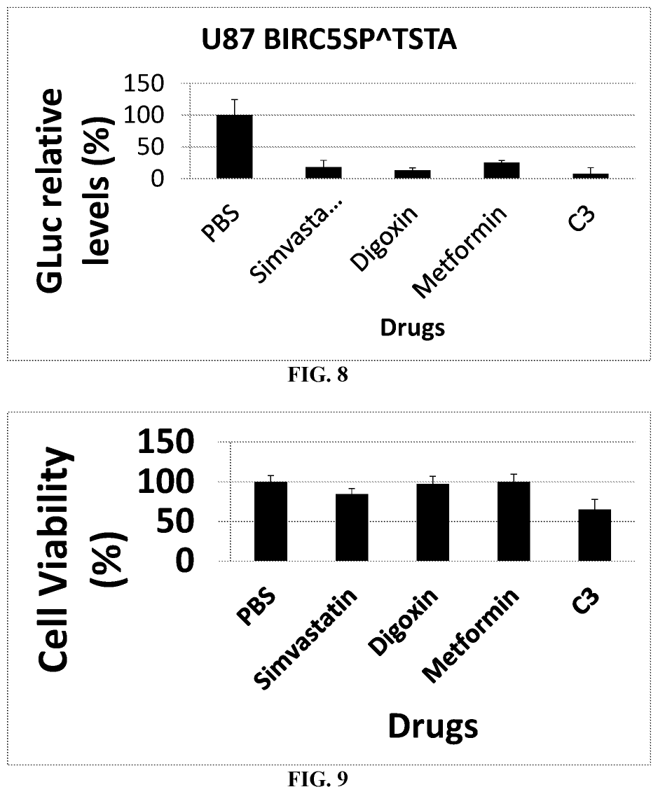

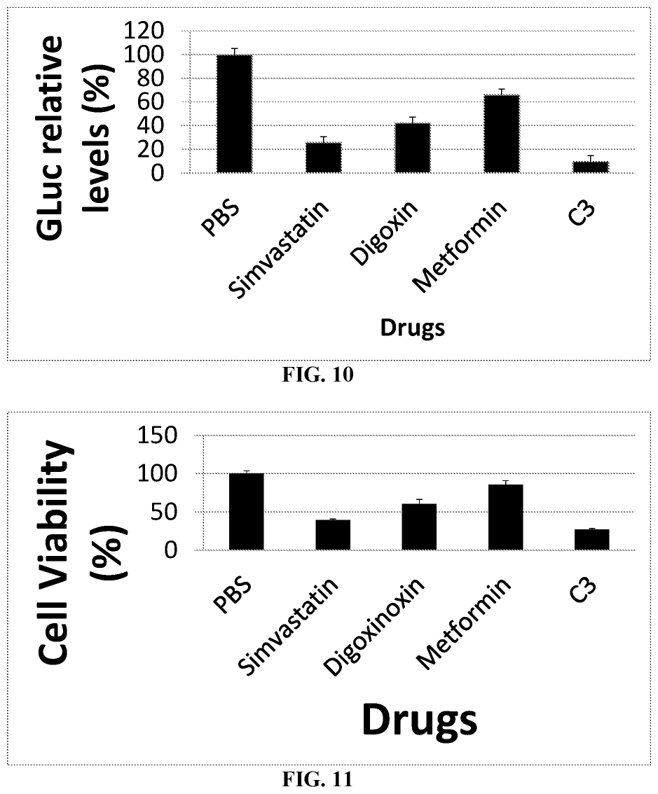

FIG. 8 is a graph that shows that C3 therapy inhibits BIRC5 promoter activity in U87 Glioblastoma Cells. FIG. 9 is a graph that shows the reduced viability of U87 Glioblastoma Cells after C3 therapy. FIG. 10 is a graph that shows that C3 therapy inhibits BIRC5 promoter activity in U251 Glioblastoma Cells. FIG. 11 is a graph that shows the reduced viability of U251 Glioblastoma Cells after C3 therapy.

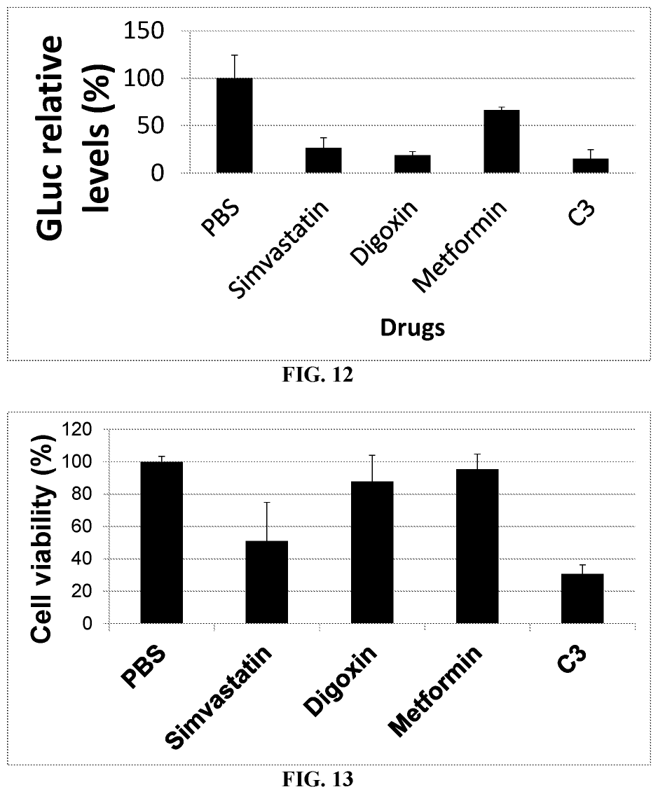

FIG. 12 is a graph that shows that C3 therapy inhibits BIRC5 promoter activity in Du145 Prostate Cancer Cells. FIG. 13 is a graph that shows the reduced viability of Du145 Prostate Cancer Cells after C3 therapy.

### Example 2. Colorectal Cancer Case Study

A colorectal patient, age 57, Caucasian female was diagnosed with a colon malignancy, subsequently diagnosed as stage III adenocarcinoma of the colon. After testing for microsatellite instability, it was determined that the patient was not a candidate for immunotherapy with pembrolizumab or nivolumab therapy. A Colon Cancer Hot Spot Panel v2 revealed NRAS wildtype, BRAF wildtype, and KRAS G12D mutation. Because of the KRAS mutation the patient was not a candidate for cetuximab or panitumumab therapy.

The patient underwent a right hemicolectomy and omentectomy, had cytoreductive surgery and intraperitoneal chemotherapy. The tumor had penetrated the surface of the visceral peritoneum, however.

The omental tissue and margins were negative for dysplasia and carcinoma. One of twenty-nine lymph nodes were positive for metastatic carcinoma with extranodal extension with the presence of lymphovascular and perineural invasion. The pathological staging was pT4a, pN1a, stage IIIC with a tumor size with a greatest dimension of 5.7 cm.

Following surgery, the patient underwent twelve cycles of adjuvant first-line chemotherapy with a combination leucovorin, fluorouracil, and oxaliplatin (FOLFOX). Positron emission tomography/computed tomography (PET/CT) showed no evidence of focal uptake with a subsequent PET/CT showing no evidence for hypermetabolic neoplasm.

A restaging PET/CT scan found multiple newly enlarged left para-aortic lymph nodes with increased fluorodeoxyglucose (FDG) uptake, metastasis and a hypermetabolic mass in the uterus. Biopsies of the endometrium and cervix revealed secondary spread of adenocarcinoma from the ileocecal primary with the adenocarcinoma infiltrating the posterior cervical wall.

The patient underwent four cycles of neoadjuvant chemotherapy with a combination of leucovorin, fluorouracil, and irinotecan (FOLFIRI). The patient also underwent a hysterectomy with bilateral salpingo-oophorectomy, partial cystectomy and lymph node resection. Pathology found metastatic adenocarcinoma of colonic origin in the posterior uterine wall, right ovary, two left pelvic lymph nodes, and one right pelvic lymph node. An additional twelve cycles of adjuvant FOLFIRI with capecitabine were provided.

Restaging PET/CT scan showed resolution of hypermetabolic uterine mass, retroperitoneal adenopathy, and pelvic adenopathy. New, mildly FDG bilateral inguinal lymph nodes were found. Maintenance chemotherapy with capecitabine and bevacizumab was provided. Subsequent PET/CT found a hypermetabolic left para-aortic lymph node with elevated carcinoembryonic antigen (CEA). The patient received stereotactic body radiation therapy (SBRT) with five total fractions delivered every other day at 40 Gray. Capecitabine was restarted but discontinued due to possible cardiotoxicity and lack of anti-tumor activity.

A restaging PET/CT showed a stable left para-aortic lymph node, but CEA increased significantly. Due to the elevated CEA and the stable para-aortic lymphadenopathy that did not respond to radiation, the patient entered Phase 1B trial of metformin, digoxin, simvastatin in subjects with advanced pancreatic cancer and other advanced solid tumors (NCT03889795).

The Phase IB trial of metformin, digoxin, and simvastatin with advanced solid tumors is an open label, single center, single arm dose escalation study. C3 (metformin, simvastatin, and digoxin) was given as three oral pills. The patient described received metformin 850 mg po/day for two weeks and 1,700 mg po/day for the next two weeks, simvastatin 20 mg po/day, and digoxin 0.25 mg.

The patient began treatment with a CEA of 23.4 ng/mL with CT imaging on showing enlarged right common iliac lymph nodes with increased presacral free fluid. Following treatment, CEA decreased to 18.2 ng/mL. The patient reported no side effects from the C3 treatment regimen. Subsequent CT scans showed stable and minimal retroperitoneal lymphadenopathy with CEA reducing to 17.4 ng/mL. No new symptoms or side effects were reported.

CT scans showed no evidence of recurrence and stable retroperitoneal lymphadenopathy. Subsequent testing and CT scans showed no CEA above 30 ng/mL and no evidence of disease progression. CT scans shows a stable appearance of the abdomen and pelvis compared with the previous scan and no new evidence of metastatic disease. The patient completed thirteen cycles of the C3 protocol treatment with no significant side effects or safety concerns. The patient showed a significant response to the treatment. CT scans showed no evidence of metastatic disease at 565 days.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of inhibiting growth of cancer in a patient, the method comprising contacting the cancer with a combination therapy that comprises: a metformin or a metformin analog; at least one statin selected from the group consisting of: simvastatin, atorvastatin, rosuvastatin, fluvastatin, lovastatin, pitavastatin, pravastatin, and prosuvastatin; and a cardiac glycoside, in amounts sufficient to inhibit the growth of the cancer, wherein the cancer is selected from breast, colorectal, glioblastoma or prostate cancer.

2. The method of claim 1, further comprising a pharmaceutically acceptable carrier selected to be compatible with metformin, simvastatin, and digoxin.

3. The method of claim 1, wherein the combination therapy is a composition formulated as a time release formulation.

4. The method of claim 1, wherein the combination therapy is a composition is disposed in a capsule, a tablet, a powder, or a liquid.

5. The method of claim 1, wherein the combination therapy comprises: 5-80 milligrams of simvastatin; 500-2550 milligrams of metformin; and 0.125-0.250 milligrams of digoxin; or 5-80 milligrams per os (po) of simvastatin; 500-2550 milligrams po of metformin; and 0.125-0.250 milligrams po of digoxin.

6. The method of claim 1, wherein the metformin, simvastatin and digoxin are combined into one or more doses before providing to the patient with breast, colorectal, glioblastoma or prostate cancer.

7. The method of claim 1, further comprising observing the breast, colorectal, glioblastoma or prostate cancer for evidence of reduced tumor size, inhibition of tumor growth, slowing of tumor growth, or tumor death following exposure to the metformin, simvastatin and digoxin.

8. The method of claim 1, wherein the colorectal cancer is selected from at least one of: familial polyposis, polyps with DNA mismatch repair, MSI, Lynch Syndrome, adenomatous polyps, or sessile serrated polyps.

9. The method of claim 1, wherein the breast, colorectal, glioblastoma or prostate cancer is a metastatic cancer.

10. The method of claim 1, wherein the patient is a human patient and is administered 500-2550 milligrams/day of metformin, 5-80 milligrams/day of simvastatin, and 0.125-0.250 milligrams/day of digoxin.

11. The method of claim 1, further comprising combining the composition with effective amounts of at least one of Bevacizumab, Irinotecan Hydrochloride, Capecitabine, Cetuximab, Ramucirumab, Oxaliplatin, Cetuximab, 5-Fluorouracil, Ipilimumab, Irinotecan Hydrochloride, Pembrolizumab, Leucovorin Calcium, Trifluridine and Tipiracil Hydrochloride, Nivolumab, Oxaliplatin, Panitumumab, Ramucirumab, Regorafenib, Trifluridine and Tipiracil Hydrochloride, Ziv-Aflibercept, or drug combinations selected from: CAPOX, FOLFIRI, FOLFIRI-BEVACIZUMAB, FOLFIRI-CETUXIMAB, FOLFOX, FU-LV, XELIRI, or XELOX.

12. A method treating a cancer identified as expressing BIRC5 mRNA, the method comprising providing a human patient with an effective amount of metformin, simvastatin and digoxin sufficient to inhibit the expression of BIRC5 mRNA or BIRC5 activity in the cancer, wherein the amounts of metformin, simvastatin, and digoxin sufficient to inhibit in vivo growth of a human cancer when administered to the patient diagnosed with cancer, wherein the cancer is selected from breast, colorectal, glioblastoma or prostate cancer.

13. The method of claim 12, further comprising a pharmaceutically acceptable carrier selected to be compatible with metformin, simvastatin, and digoxin.

14. The method of claim 12, wherein the metformin, simvastatin and digoxin are formed as a time release formulation.

15. The method of claim 12, wherein the metformin, simvastatin and digoxin are disposed in a capsule or tablet.

16. The method of claim 12, wherein amounts of metformin, simvastatin, and digoxin sufficient to inhibit in vivo growth of a human breast, colorectal, glioblastoma or prostate cancer cell when administered to a patient diagnosed with breast, colorectal, glioblastoma or prostate cancer.

17. The method of claim 12, wherein the metformin, simvastatin and digoxin comprises: 5-80 milligrams of simvastatin; 500-2550 milligrams of metformin; and 0.125-0.250 milligrams of digoxin; or 5-80 milligrams per os (po) of simvastatin; 500-2550 milligrams po of metformin; and 0.125-0.250 milligrams po of digoxin.

18. The method of claim 12, wherein the metformin, simvastatin and digoxin are combined into one or more doses before providing to the human patient with breast, colorectal, glioblastoma or prostate cancer.

19. The method of claim 12, further comprising observing the breast, colorectal, glioblastoma or prostate cancer for evidence of reduced tumor size, inhibition of tumor growth, slowing of tumor growth, or tumor death following exposure to the metformin, simvastatin and digoxin.

20. The method of claim 12, wherein the colorectal cancer is selected from at least one of: familial polyposis, polyps with DNA mismatch repair, MSI, Lynch Syndrome, adenomatous polyps, or sessile serrated polyps.

21. The method of claim 12, wherein the breast, colorectal, glioblastoma or prostate cancer is a metastatic cancer.

22. The method of claim 12, wherein the patient is administered 500-2550 milligrams/day of metformin, 5-80 milligrams/day of simvastatin, and 0.125-0.250 milligrams/day of digoxin.

23. The method of claim 12, further comprising combining a population of colorectal cancer cells with amounts of at least one of Bevacizumab, Irinotecan Hydrochloride, Capecitabine, Cetuximab, Ramucirumab, Oxaliplatin, Cetuximab, 5-Fluorouracil, Ipilimumab, Irinotecan Hydrochloride, Pembrolizumab, Leucovorin Calcium, Trifluridine and Tipiracil Hydrochloride, Nivolumab, Oxaliplatin, Panitumumab, Ramucirumab, Regorafenib, Trifluridine and Tipiracil Hydrochloride, Ziv-Aflibercept, or drug combinations selected from: CAPOX, FOLFIRI, FOLFIRI-BEVACIZUMAB, FOLFIRI-CETUXIMAB, FOLFOX, FU-LV, XELIRI, or XELOX.

24. The method of claim 12, wherein the metformin, simvastatin and digoxin is combined with a plurality of cells in an amount sufficient to promote cellular apoptosis in the breast, colorectal, glioblastoma, or prostate cancer.

25. The method of claim 12, further comprising treating the patient with gemcitabine.

26. A method treating a cancer in a human patient identified as expressing BIRC5 mRNA, the method comprising:
determining that cancer cells in a subject have an overexpression, or increased activity, of BIRC5 when compared to a subject that does not have cancer; and
providing the patient with an effective amount of metformin, simvastatin and digoxin sufficient to inhibit the expression of BIRC5 mRNA or BIRC5 activity in the cancer, wherein the amounts of metformin, simvastatin, and digoxin sufficient to inhibit in vivo growth of a human cancer when administered to the patient diagnosed with cancer, wherein the cancer is selected from breast, colorectal, glioblastoma or prostate cancer.
